(19) United States
(12) Patent Application Publication
(10) Pub. No.: US 2022/0192901 A1
(43) Pub. Date: Jun. 23, 2022

(54) EFFICIENTLY MANUFACTURED POLYHEDRON CONTAINER ADAPTED FOR DISPLAY OF CONTENTS THROUGH PLURALITY OF ADJACENT OUTER PANELS

(71) Applicant: The Procter & Gamble Company, Cincinnati, OH (US)

(72) Inventors: Jerry Ray STEPHENS, Hamilton, OH (US); Taylor Javier MORRIS, Cincinnati, OH (US)

(21) Appl. No.: 17/551,313

(22) Filed: Dec. 15, 2021

Related U.S. Application Data
(60) Provisional application No. 63/126,603, filed on Dec. 17, 2020.

(51) Int. Cl. A61F 13/551; B65D 5/42; B65D 5/54; B65D 77/04; B65D 5/02

(52) U.S. Cl. CPC A61F 13/5514; B65D 5/4204; B65D 2577/047; B65D 77/0413; B65D 5/0227; B65D 5/542

(57) ABSTRACT

A container including an assembled combination of opaque paperboard and substantially transparent polymer sheet is disclosed. The containing may have a convex polyhedron shape and include at least a first group of three substantially flat mutually adjacent outer panels, wherein the first group of mutually adjacent outer panels share at least a first substantially transparent window that is present on and extends continuously and wraps from a first of the first group of panels, to a second of the first group of panels, to a third of the first group of panels, the first window being defined by a first window cutout through the paperboard, and the first window being occupied by a continuous section of the polymer sheet present and at least partially forming each of the first group of panels. The container may be a retail package.

### CROSS REFERENCE TO RELATED APPLICATION

This application claims the benefit of U.S. Provisional Application No. 63/126,603, filed Dec. 17, 2020, the substance of which is incorporated herein by reference.

### BACKGROUND

Manufacturers and sellers of retail products typically sold as packages containing grouped quantities of individually consumable products continuously strive to design retail packaging containers with features that end-purchasers will find eye-catching, attractive and, at the same time and to the extent feasible, functionally advantageous. Given the myriad combinations of design options and component materials available for configuring retail containers, there is always room for improvement in designs for containers toward the objectives of providing the manufacturer/seller a competitive advantage in (1) attracting end-purchasers to view and consider their products when displayed in a retail setting; (2) providing functional advantages for storage and dispensation of the individually consumable products following purchase, and (3) providing for efficient and cost-effective manufacture.

### DESCRIPTION OF THE FIGURES

FIG. 1 is a perspective view of an example of a container, in this example, having a convex polyhedral shape, more particularly in this example a rectangular cuboid shape having six panels forming six sides.

FIG. 2A is a plan view of an example of a container flat, outward-facing surfaces facing the viewer.

FIG. 2B is a plan view of another example of a container flat, outward-facing surfaces facing the viewer.

FIG. 3 is a view of the container of FIG. 1, with the addition of windows.

FIG. 4 is a view the container of FIG. 3, expanded to show additional detail.

FIG. 5 is a front view of the container of FIGS. 3 and 4 depicted as it might appear containing a plurality of examples of individually consumable products.

FIG. 6 is a front view of an example of a container having windows as it might appear containing a plurality of examples of individually consumable products, identifying and locating an example of an opening feature that might be included on/in the container.

FIG. 7 is a rear view of the example of FIG. 6, identifying and locating an example of an opening feature that might be included on/in the container.

FIG. 8 is a view of the example of the container shown in FIG. 7, depicting opening of the container via the opening feature.

FIG. 9 is a side view of the container of FIG. 8, depicting opening of the container via the opening feature.

FIG. 10 is a front view of an example of a container having windows as it might appear containing a plurality of examples of individually consumable products, the products bearing examples of decorative graphics forming a coherent design across viewable surfaces of the plurality of products.

FIG. 11 is a view of an example of an individually packaged product that might be contained within a container.

FIG. 12 is a view of the example of FIG. 11 having an example of a closure tape or tab.

FIG. 13 is a rear view of an example of a container, identifying and locating an example of an opening feature that might be included on/in the container, the opening feature including a closure tab.

FIGS. 14A-14G depict respective pairs of examples of visually similar pairs of profiles of respective opening flaps of a container and a package that may be contained in the container.

FIGS. 15A and 15B are schematic front and side views, respectively, of fixture components used in the Bending Resistance Measurement Method described herein.

### DESCRIPTION OF EXAMPLES

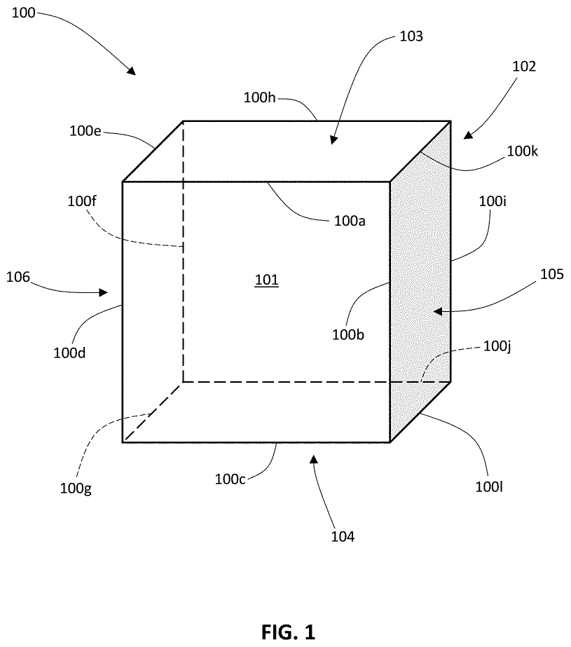

Referring to FIG. 1, a container 100 within contemplation of the present disclosure may have a combination of substantially flat outer surfaces that together form any convex polyhedral shape, but in the depicted non-limiting example the shape may be a rectangular cuboid, or “box”. Containers having a rectangular cuboid shape are used for transporting, packaging and displaying many types of products, and are relatively efficiently manufactured. The container 100 may have a variety of construction/assembly configurations and a variety of configurations to provide for opening of the container and access to its contents. A container having a rectangular cuboid shape will having the following 12 outwardly-protruding corners and six panels forming outward-facing surfaces, identified in FIG. 1 herein for reference:

| | |
|---|---|
| top-front corner 100a; | bottom-rear corner 100j; |
| right-front corner 100b; | top-right corner 100k; |
| bottom-front corner 100c; | bottom-right corner 100l; |
| left-front corner 100d; | front panel 101; |
| top-left corner 100e; | rear panel 102; |
| rear-left corner 100f; | top panel 103; |
| bottom-left corner 100g; | bottom panel 104; |
| top-rear corner 100h; | right side panel 105; and |
| right-rear corner 100i; | left side panel 106. |

For purposes herein, front panel 101 will be deemed the facing panel of a container to be used as a retail package, i.e., the panel that is configured to be displayed facing forward in a typical physical retail setting, e.g., when the container is sitting on a retail store shelf.

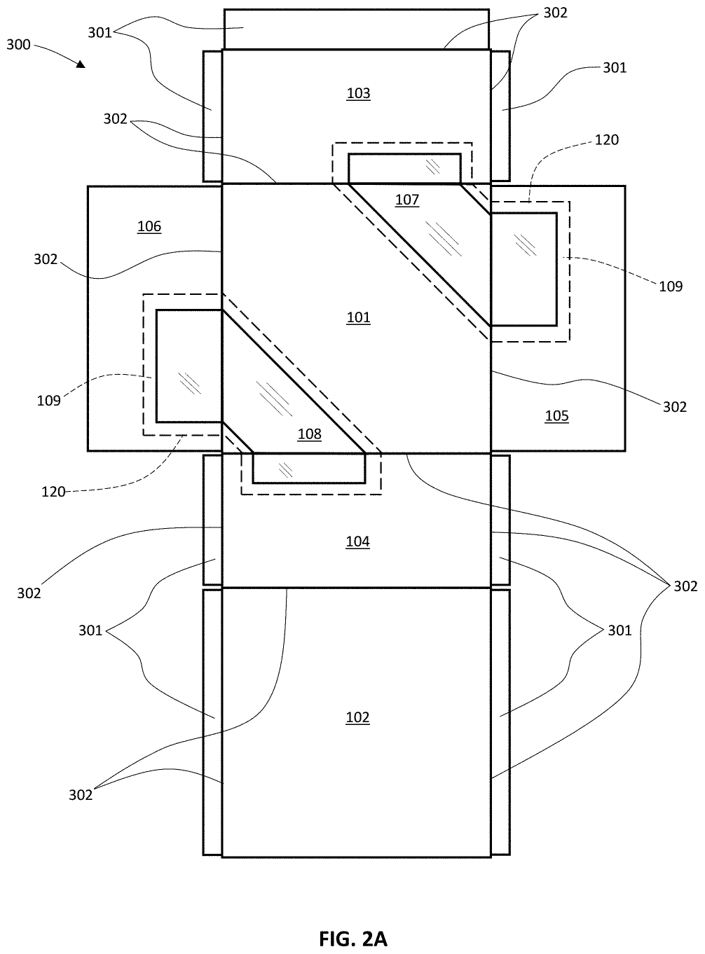

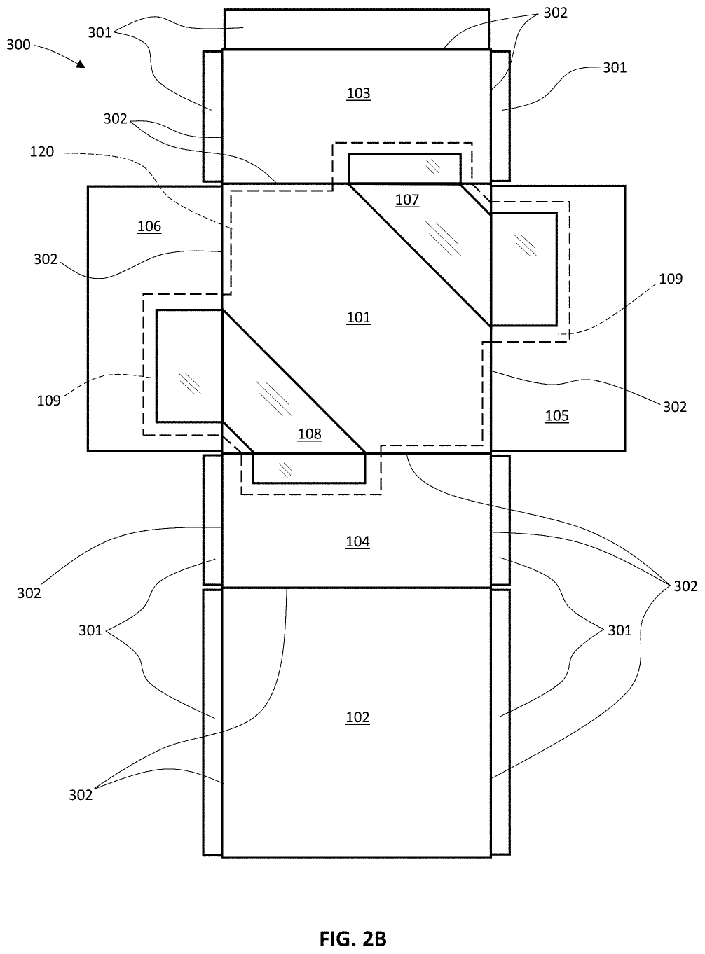

Referring to FIGS. 1 and 2, in some examples, container 100 may be assembled from a unitary, suitably configured flat 300, which may be cut from a sheet of paperboard or cardboard, or any other suitable sheet material having sufficient rigidity to form a structurally stable container suited to it intended purposes. In some examples, it may be preferred that the assembled container 100 be assembled so that it may not be opened non-destructively, so as to discourage premature opening or tampering prior to purchase of the container (with contained product) by a consumer. Accordingly, in some examples container 100 may be assembled from a flat 300 that includes a configuration of assembly flaps 301 that are folded about corners along assembly fold lines 302 and underlap sections forming one or more of adjacent panels 101-106, wherein a respective overlying panel and underlying flap 301 are bonded to each other, e.g., via adhesive, and form a corner of the container at the assembly fold line 302, as well as impart structural integrity to the container. Folding about fold lines 302 may be facilitated by embossing or impressing grooves in the material of the flat along the fold lines, during its manufacture, thereby reducing bending stiffness/bending resistance at the fold lines. In some examples, at least two opposing panels of the container 100 may have an assembly flap 301 underlying and bonded thereto. In some examples, at least two opposing panels of the container 100, and one panel adjacent to each of the opposing panels, the one panel meeting each of the opposing panels at respective corners, may have an assembly flap 301, or a plurality of assembly flaps 301, underlying the one adjacent panel. It will be appreciated that, in the example depicted in FIGS. 2 and 3, following assembly of a container 100 from the depicted container flat 300, each of opposing panels 105, 106 will have assembly flaps 301 underlying them, those assembly flaps extending upward and downward (relative the orientation shown in FIG. 1) from top-left and top-right corners of top and bottom panels 103, 104. Additionally, following assembly of the container rear panel 102 will be adjacent panels 105, 106, and will also have at least one assembly flap 301 underlying it, that assembly flap extending downward from top panel 103 at the top-rear corner 100h of the container 100.

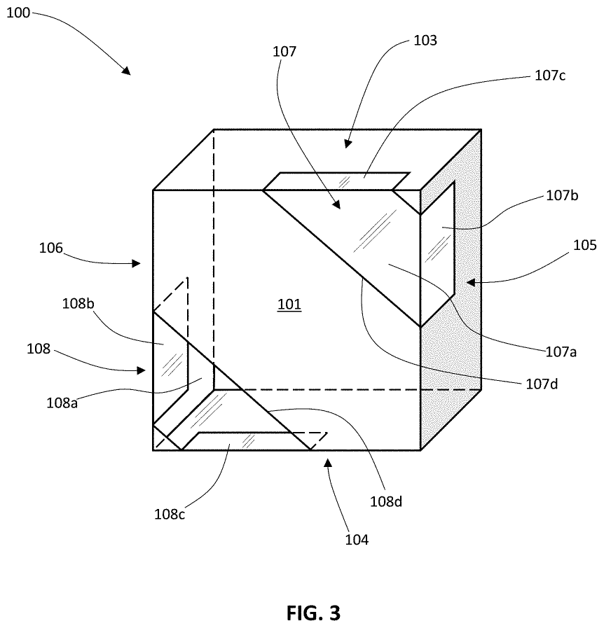

Referring to FIGS. 2A, 2B, 3 and 4, in some examples, container 100 may be imparted with one or more translucent or transparent windows 107, 108. Such windows may be formed by cutting associated holes of the desired window shape(s) through the material (e.g., paperboard) forming the main portion of the container flat 300, along window cutouts 107d, 108d, and then overlaying over each such hole on the inside surface (relative the finished container) of the flat, a suitably shaped and sized section of a translucent or transparent window sheet material 120, with sufficient margins 109 of window sheet material about the window cutout edges to provide suitable regions of interface between these materials to facilitate affixing the window sheet material 120 to the main portion material via, for example, adhesive disposed between the window sheet and main portion material in the margins 109. A single integral section of the window sheet material 120 may extend across several panels of the flat 300 as shown, and be folded into 90-degree bends along with the main material of the flat along fold lines 302, to form corners of the container. The window sheet material 120 may be suitably sized and shaped as a single continuous section so as to extend across and cover all of the window cutouts in the flat 300 (as suggested in FIG. 2B), or alternatively, window sheet material 120 may be suitably sized and shaped in a plurality of individual sections, so to only cover each window cutout individually (as suggested in FIG. 2A). Where a plurality of windows are present on a single panel as in the depicted examples, it may be preferred that the window sheet material 120 be a single continuous section as suggested in FIG. 2B, such that it imparts added rigidity and structural robustness to the portion(s) of the panel between the window cutouts. On the other hand, if the material (e.g., paperboard) from which the main portion of the flat 300 is made is sufficiently rigid to provide a suitable structurally robust container even after being cut to create the desired configuration of windows, it may be desired to include individual sheets more closely sized and shaped only for each window, for purposes of saving material costs of window sheet material.

The selected window sheet material may be any translucent or transparent film or sheet material, and may be sheet material extruded or cast from a thermoplastic polymeric resin. In some examples, the selected resin may be polyethylene terephthalate (PET) or modified PET; in other examples, the selected resin may be polypropylene. In order to retain structural integrity in the assembled container 100 despite removal of material within the window cutout(s) from the main portion of the flat, to form the windows, in some examples, it may be desired that the window sheet material selected have a rigidity, reflected as Bending Resistance, that is at least 30 percent, more preferably at least 40 percent, and even more preferably at least 50 percent, of that of the material forming the main portion of the container flat, measured according the Bending Resistance Measurement Method set forth below. PET may be particularly suited to this purpose, in that sheets extruded or cast from PET tend to be relatively stiffer, per unit caliper, than sheets extruded or cast from other translucent/transparent thermoplastic resins, while being relatively inexpensive and easily processed and worked. Additionally, although relatively stiff, sheets cast or extruded from PET tend to be flexible and tough, not brittle, and plasticly deformable in folding processes, making them amenable to easy creation of folds therein, to form robust corner structures. Further, PET-based material may be selected such that sheets cast or extruded from it tend to have optical properties that are desirable for windows, i.e, near or substantial colorlessness and high clarity/transparency.

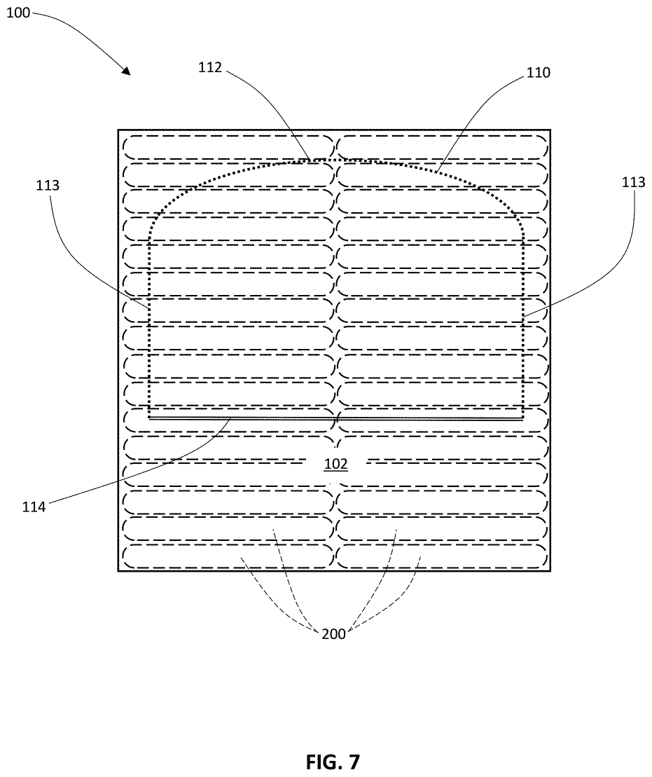

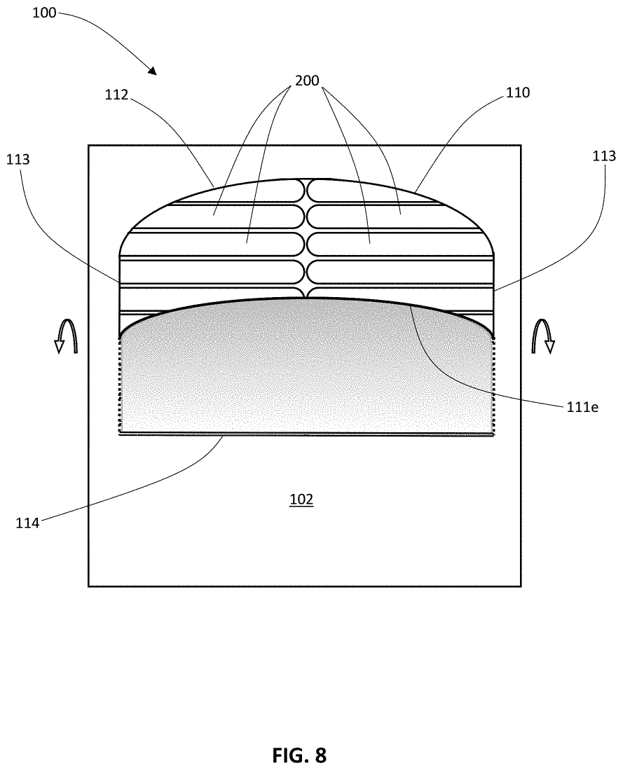

In the examples depicted in FIGS. 2A, 2B, and 3-9, (and using the references to panels and corners identified in FIG. 1) it can be seen that flat 300 forming container 100 may be configured such that a window such as windows 107, 108 occupies three mutually adjacent panels. In the example shown in the figures, and referring particularly to FIG. 3, a first window 107 may occupy front panel 101 (window portion 107a), and wrap around to mutually adjacent top panel 103 (window portion 107c) and mutually adjacent right side panel 105 (window portion 107b). A second window 108 may occupy front panel 101 (window portion 108a), and wrap around to mutually adjacent bottom panel 104 (window portion 108c) and mutually adjacent left side panel 106 (window portion 108b). At the same time, it can be seen that the window 107 does not extend to the tri-corner intersection of top-front corner 100a, right-front corner 100b and top-right corner 100k; and window 108 does not extend to the tri-corner intersection of bottom-front corner 100c, left-front corner 100d, and bottom-left corner 100g. Further, it will be observed that the edges of the main/largest portions of the windows 107a, 108a may be angled or slanted, relative the corners outlining the panel 101 on which they are situated. The configuration illustrated provides several advantages: (1) It provides a view of product(s) contained within the container from three sides of the container; (2) it avoids a construction wherein edges of the window sheet material are exposed, in ordinary handling and opening of the container; (3) despite providing windows that are cut through the main sheet material forming the container flat 300, the assembled container 100 has inherent structural integrity, particularly when selected window sheet material has suitable rigidity; (4) the configuration is relatively easily manufactured and assembled into a container.

With regard to item (2), when relatively rigid window sheet materials such as those made of PET are cut, the cut edges can be sharp enough to be uncomfortable, or even cause skin cuts, to a consumer who runs their hands along them. Accordingly, it is advantageous to configure a container with windows that incorporate such materials such that no cut edges of the window sheet material project or extend away from the paperboard material, upon opening of the container 100 in the designed manner.

With regard to item (3), when the window sheet material is plasticly deformable in the manner of sheet material formed of PET, structurally well-defined and rigid corners in the material may be formed at, e.g., corners 100a, 100b, 100c and 100d that substantially retain the structural integrity of the container as if no windows were present.

With regard to item (4), it may be noted that no portion of the window sheet material is present in any of the assembly flaps, and thus, does not complicate the folding thereof or assembly of the container 100 from the flat 300.

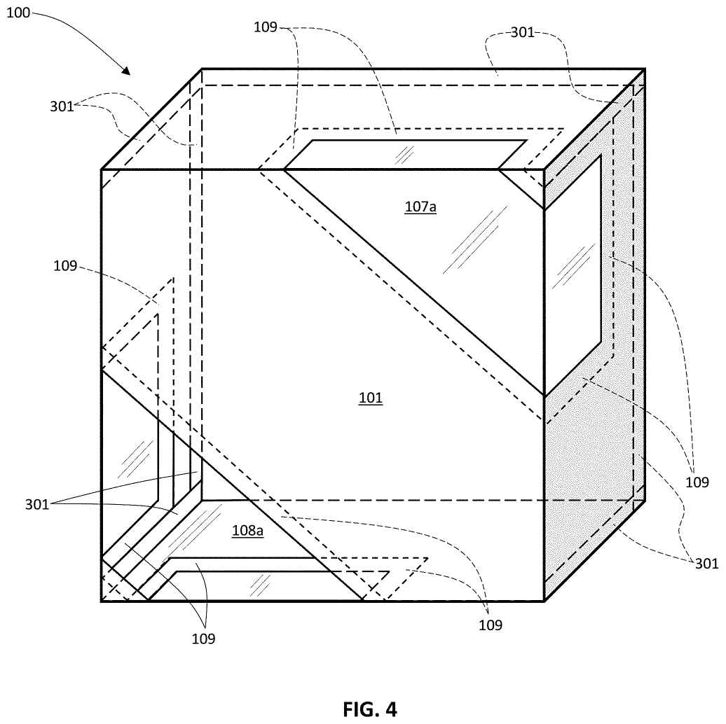

Referring now to FIG. 5, the container 100 in the examples depicted may be particularly well-suited to contain, and visibly display, one or more orderly stacks of individually consumable products 200, along at least three surfaces of the container. This may be deemed useful when the individually consumable products are themselves deemed attractive and/or attractively individually packaged and the manufacturer therefore wishes to display them in a retail setting, and/or, such visibility is useful to the consumer, following purchase and during storage/dispensation, to enable the consumer to monitor the quantity of individually consumable products remaining in the supply within the container 100, as the products are consumed, while at the same time, the remaining unconsumed individually consumable products remain neatly contained within the container 100.

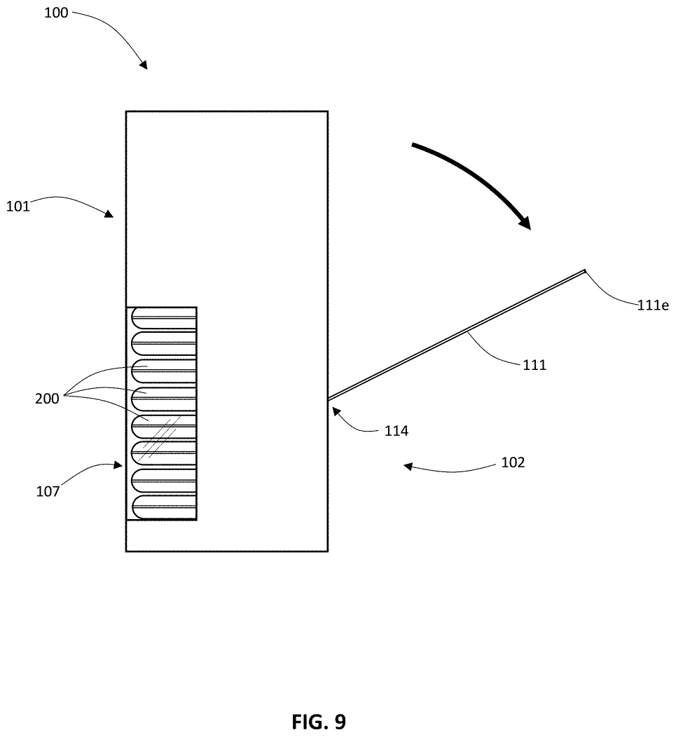

Referring to FIG. 10, the manufacturer may provide individually consumable products 200 in one or more orderly stacks, wherein each product in a stack bears an individual configuration of decorative graphics 201a, and wherein when the products are stacked their individual configurations of decorative graphics 201a collectively form a coherent design recognizable across a plurality of the products' viewable surfaces in the stack, the coherent design being visible through at least one window 107, 108. One example of such a coherent design may be seen in FIG. 10.

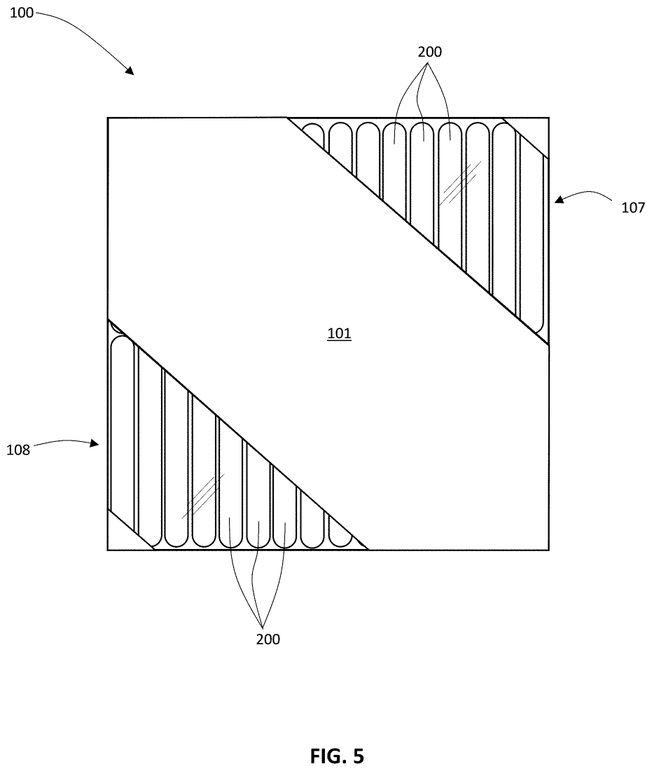

Referring to FIGS. 6-9 (and using the references provided by FIG. 1), in some examples a panel that does not include a window, such as rear panel 102, may include an access opening configuration. The access opening configuration in some examples may include a path of weakness 110 configured to facilitate tearing of the material of the panel therealong, providing an opening in the container and providing access to its contents. The path of weakness may be created by a path of perforations or scoring in the material of the panel. The path of weakness may be configured such that an opening flap 111 is formed, following tearing therealong. In some examples the path of weakness may include opposing straight portions 113 and a transverse portion 112 joining the straight portions 113. The access opening configuration may also include a flap hinge line 114, along which the material of the panel has been embossed or grooved, to promote bending of the material along the hinge line 114 such that the flap may be neatly opened and hinge about line 114 in manner similar to the way in which a door opens about its hinges.

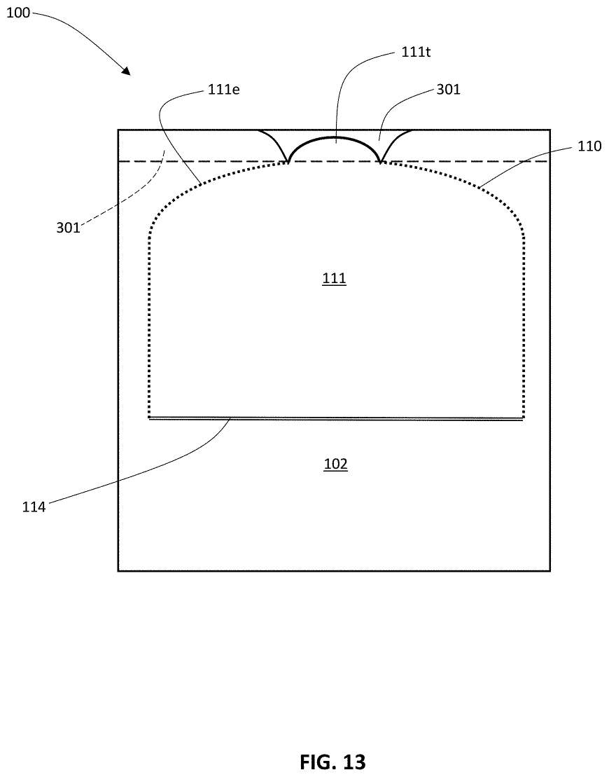

An opening flap may be configured to include a reclosing feature. Referring to FIG. 13, in the example shown, the reclosing feature may include a closure tab 111t. The container 100 may include another feature configured to interact with a closure tab, such as a slot (not specifically shown) cut through the panel on which the opening flap 111 is located, positioned and sized to enable the closure tab 111 to be inserted therein, in a position in which it will hold flap 111 in a reclosed position. In another example specifically shown in FIG. 13, the flap 111 with included closure tab 111t may be positioned and arranged, and the tab may be precut from the panel 102 as shown, such that the tab may be inserted behind an assembly flap 301 underlying panel 102, thereby holding flap 111 in a reclosed position following opening/tearing of the package along path of weakness 110 and resulting creation of the opening flap 111.

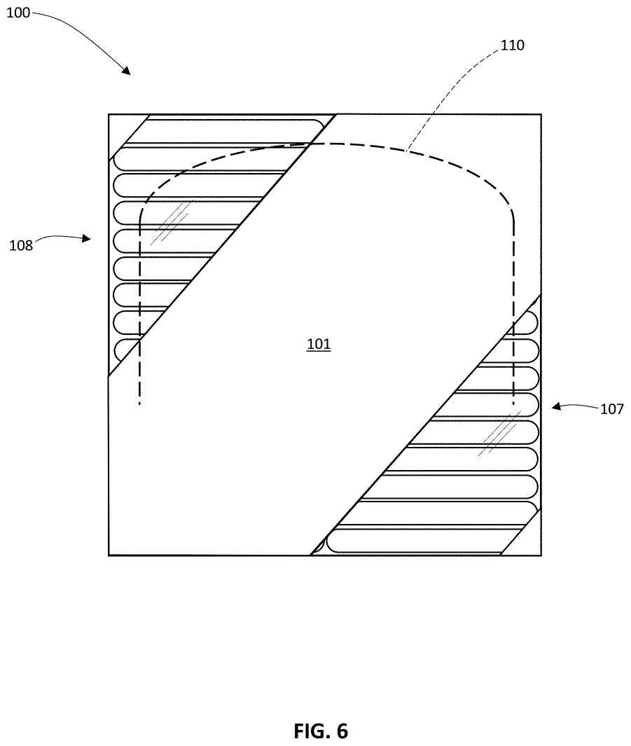

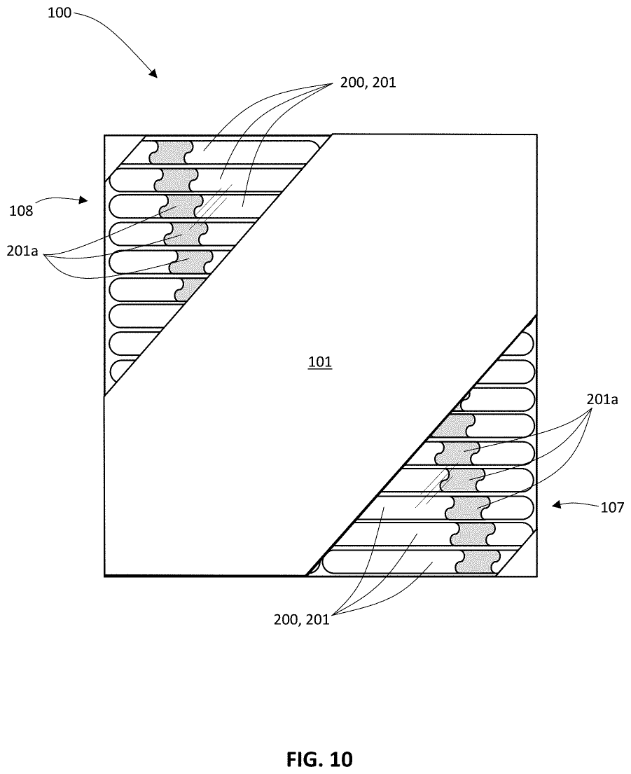

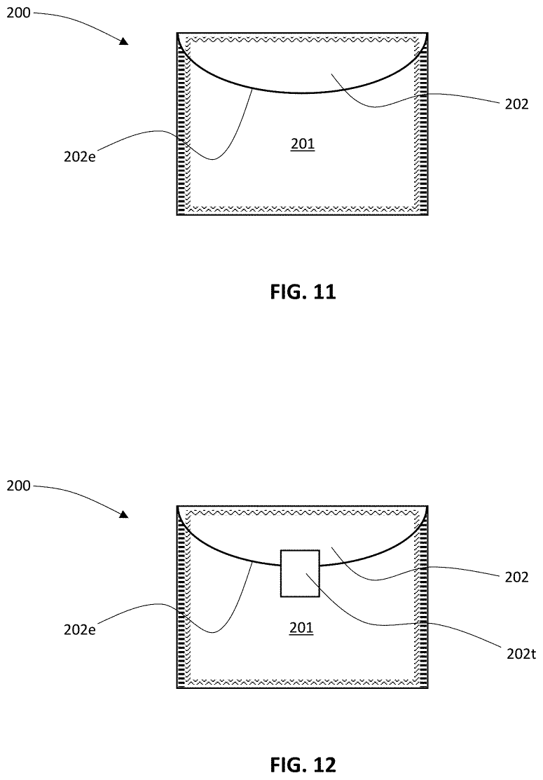

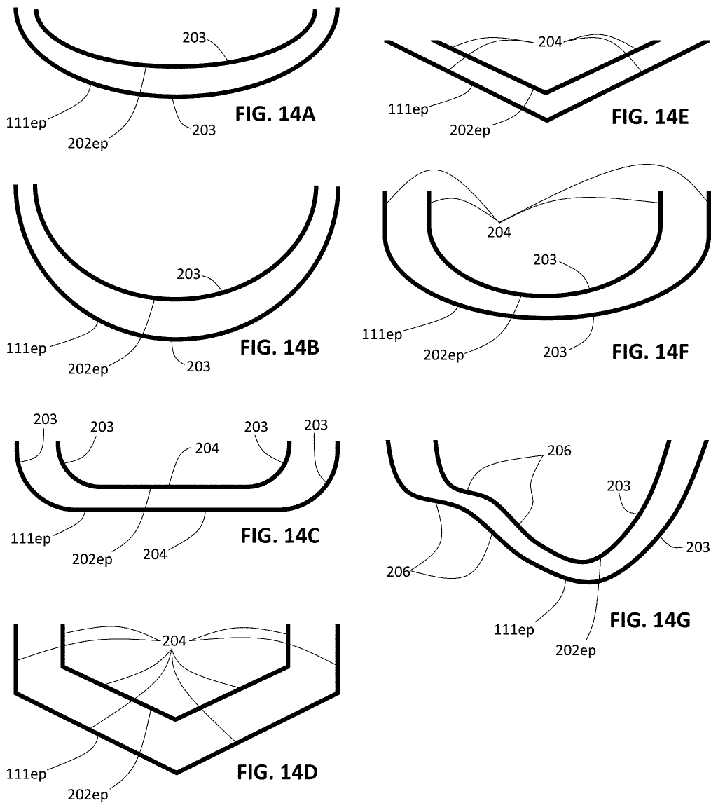

Referring now to FIGS. 7, 8, 11, 12 and 14A-14G, individually consumable products 200 may by individually packaged (e.g., wrapped), in individual packages 201 (FIGS. 11, 12). Individual packages 201 may also include opening flaps. In some examples, packages 201 may have the form of an envelope with an opening/closing flap 202. It has been learned that configuring a container opening flap edge 111e with a container opening flap edge profile 111ep, and an individual package flap edge 202e with a package flap edge profile 202ep (non-limiting examples shown in FIGS. 14A-14G), such that the two profiles 111ep and 202ep are visually similar, has associative effects believed to be appreciated by consumers, in some cases being perceived as suggesting that the container and the individual packages therein are opened in a similar manner. Herein, “visually similar” means that the respective profiles 111ep and 202ep trace respective paths that:
(a) have the same number of straight line segments such as line segments 204; and/or
(b) have the same number of continuous curves such as curves 203, wherein the curves have the same number of inflection points such as inflection point 206.
   Herein, a “continuous curve” is a portion of a profile that is not a straight line segment, and does not have a break, i.e., an abrupt change of direction. Examples of visually similar pairs of respective profiles 111ep and 202ep are illustrated in FIGS. 14A-14G. The illustrated pairs meet the definition of “visually similar” set forth above, as follows:

FIGS. 14A, 14B: Profiles 111ep and 202ep each have the same number (one) of continuous curves 203, with the same number of inflection points (zero).

FIG. 14C: Profiles 111ep and 202ep each have the same number (one) of straight line segments 204. This alone is sufficient to cause the profiles to meet the above definition of “visually similar.” However, further, profiles 111ep and 202ep each have the same number (two) of continuous curves 203, with the same number of inflection points (zero). This alone is also sufficient to cause the profiles to meet the above definition of “visually similar.”

FIG. 14D: Profiles 111ep and 202ep each have the same number (four) of straight line segments 204.

FIG. 14E: Profiles 111ep and 202ep each have the same number (two) of straight line segments 204.

FIG. 14F: Profiles 111ep and 202ep each have the same number (two) of straight line segments 204. This alone is sufficient to cause the profiles to meet the above definition of “visually similar.” However, further, profiles 111ep and 202ep each have the same number (one) of continuous curves 203, with the same number of inflection points (zero). This alone is also sufficient to cause the profiles to meet the above definition of “visually similar.”

FIG. 14G: Profiles 111ep and 202ep each have the same number (one) of continuous curves 203, with the same number of inflection points 206 (two).

Referring to FIGS. 12 and 13, for purposes herein, a closure tab such as closure tab 111t, or closure tape such as closure tape 202t, that outlines an extension from the flap profile 111ep, 202ep that creates an opposing pair of abrupt discontinuities (abrupt changes of direction) from remainder of the flap profile, is disregarded when determining visual similarity of the respective remainders of the flap profiles. Thus, the flap edges 202e and 111e respectively shown in FIGS. 12 and 13 are “visually similar” as defined herein (because they have the same number of continuous curves), disregarding tab 111t and tape 202t.

The container configuration herein may be particularly well-suited to containing, displaying and visibly storing stacks of individually consumable products each having a relatively flat configuration. In some examples such products may be disposable absorbent articles, in more particular examples, feminine hygiene pads. A supply including a plurality of individual feminine hygiene pads will often be packaged by the manufacturer in a single container. When in an opened configuration ready for use, a feminine hygiene pad is typically generally flat, and accordingly, a pad may be folded into a relatively flat folded configuration of reduced surface area, then packaged in an individual wrapper. Manufacturers often seek to make the individual wrappers visually appealing to consumers. Consequently, a supply of individually folded and wrapped feminine hygiene pads can accommodate orderly stacking thereof, in one or more stacks which may be neatly contained within a container 100 having a suitably selected size, shape and interior volume therewithin. One or more windows on the container having features described herein allow consumers to view attractively packaged individual products and orderly stacks thereof, enhancing the appearance of the container when on display at retail. Following purchase, the one or more windows can serve to enable the purchaser to monitor the quantity of unconsumed individual products (e.g., pads) remaining in the container during storage, as the products are individually removed from the container for use.

Bending Resistance Measurement Method

The Bending Resistance of a sample of container material is measured using a three-point bend test on a constant rate of extension tensile tester (a suitable instrument is the MTS Alliance using Testworks 4.0 Software or TestSuite Software, as available from MTS Systems Corp., Eden Prairie, Minn.) using a load cell for which the forces measured are within 1% to 99% of the limit of the cell. All testing is performed in a room controlled at 23° C.±3° C. and 50%±2% relative humidity.

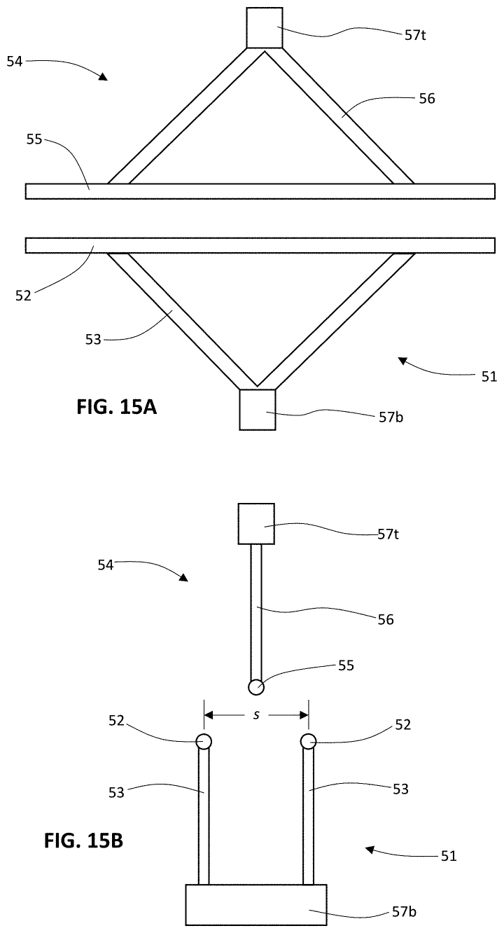

The three-point bend fixture components are described as follows, referring to FIGS. 15A and 15B.

The bottom, stationary fixture 51 consists of two cylindrical bars 52. Each is 3.15 mm in diameter by 114 mm in length, made of polished stainless steel and is mounted on a support structure 53. These two bars are mounted horizontally, parallel, at the same height and with ends aligned. The fixture is configured such that the distance between the two bars 52 (center to center, measured perpendicular to their longitudinal axes) is set at, or adjustable to, a spacing s of 25 mm±0.5 mm.

The top fixture 54 consists of a third cylindrical bar 55, also 3.15 mm in diameter by 114 mm in length, made of polished stainless steel and mounted on a support structure 56. When in place the bar 55 of the top fixture 54 is parallel, and with ends aligned, with the bars 52 of the bottom fixture 51.

The respective bottom and top fixtures are configured such that bar 55 is, or through adjustment may be, centered over bars 52.

Each of the top and bottom fixtures includes an integral adapter 57t, 57b, configured to be mounted on the upper and lower portions of tensile tester such that the longitudinal axes of the bars 52, 55 are orthogonal to the (vertical) motion of the crossbeam of the tensile tester.

Obtain samples of sizes sufficient to provide the specimens described below, of the materials from which the subject container is made. Condition the samples at 23° C.±3° C. and 50%±2% relative humidity two hours prior to testing.

Specimens are cut from areas of the sample that are free of folds, wrinkles or adhesive.

For a sample that is known to be isotropic in bending resistance, rectangular specimens are cut therefrom to a dimension of 25.0 mm by 50.0 mm, noting the side of the material that faces (or is intended to face) outwardly on a container.

For a sample that is identified as or suspected of being anisotropic in bending resistance, the objective is to determine the direction along which bending resistance of the sample is lowest. Begin by cutting a supply of circular starting specimens all of the same diameter, at least 38.0 mm but not more than 50.0 mm (a circular cutting die of the selected size may be used), from the sample, noting their rotational orientations relative the sample prior to removing them from the sample. Using the testing procedure described below and with the midpoint of each circular starting specimen centered over the lower bars 52, test successive circular starting specimens (applying only one bend per specimen) by iteratively bending successive specimens along differing axes to identify the direction along which bending resistance of the sample is lowest, and identify the circular starting specimen which exhibited such lowest bending resistance with a line identifying such direction (the identifying line will be coincident with the line of contact made on the specimen by upper bar 55, at the outset of bending). Cut final rectangular test specimens from remaining portions of the sample(s), with dimensions of 25.0 mm by 50.0 mm, with the shorter (25.0 mm) sides parallel to the identifying line marked on the identified circular starting specimen (when the identified circular starting specimen is placed in the same position and rotational orientation that it occupied prior to being cut from the sample). Again, note the side of the material that faces (or is intended to face) outwardly on a container.

Program the tensile tester for a flexural bend test, to move the crosshead such that the top fixture moves down with respect to the lower fixture at a rate of 1.0 mm/sec until the upper bar 55 touches the top surface of the specimen, then continue for an additional 12 mm collecting force (N) and displacement (mm) data at 50 Hz, and return the crosshead to its original gage. Ensure that the gap between the bars 52 of the lower fixture is 25 mm±0.5 mm (center of bar to center of bar) with the upper bar 55 centered over the lower bars.

For each test, load the specimen such that it spans the two lower bars 55, and is centered under the upper bar 55 with its longer (50.0 mm) sides perpendicular to the bars and the side identified as the outwardly-facing side (when the material forms a container) facing up. Zero the crosshead and load cell. Start the run and collect data.

Construct a graph of force (N) versus displacement (mm). From the graph, record the maximum peak force to the nearest 0.01 N. In like fashion, repeat the entire test sequence for a total of 6 test specimens.

Calculate the arithmetic mean of the peak force recorded for each set of replicates and report as Bending Resistance to the nearest 0.01 N.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments within contemplation of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

Non-limiting examples of structures and combinations of features contemplated herein include those set forth in the following claims.

What is claimed is:

1. A retail package comprising a container comprising an assembled combination of opaque paperboard and substantially transparent polymer sheet, having a convex polyhedron shape and comprising at least a first group of three substantially flat mutually adjacent outer panels, wherein the first group of mutually adjacent outer panels share at least a first substantially transparent window that is present on and extends continuously and wraps from a first of the first group of panels, to a second of the first group of panels, to a third of the first group of panels, the first window being defined by a first window cutout through the paperboard, and the first window being occupied by a continuous section of the polymer sheet present and at least partially forming each of the first group of panels.

2. The retail package of claim 1 wherein the container has a substantially rectangular cuboid shape, having six substantially flat outer panels comprising a front panel, a rear panel, a top panel, a bottom panel and left and right side panels.

3. The retail package of claim 2 wherein
the front panel has a perimeter defined by
a top-front corner,
a right-front corner,
a bottom-front corner, and
a left-front corner;
the left side panel has a perimeter defined by
a top-left corner,
a rear-left corner,
a bottom-left corner, and
the left-front corner
the rear panel has a perimeter defined by
a top-rear corner,
a right-rear corner,
a bottom-rear corner, and
the rear-left corner;
the right side panel has a perimeter defined by
a top-right corner,
the right-rear corner,
a bottom-right corner, and
the right-front corner;
the top panel has a perimeter defined by
the top-front corner,
the top-left corner,
the top-rear corner, and
the top-right corner;
the bottom panel has a perimeter defined by
the bottom-front corner,
the bottom-left corner,
the bottom-rear corner, and
the bottom-right corner;
wherein the first window occupies the top-front corner and at least one of the left-front corner and the right-front corner.

4. The retail package of claim 2 wherein a second group of three mutually adjacent outer panels share a second substantially transparent window that is present on and extends continuously and wraps from a first of the second group of panels, to a second of the second group of panels, to a third of the second group of panels, the second window being defined by a second window cutout through the paperboard, and the second window being occupied by a continuous section of the polymer sheet present at and at least partially forming each of the second group of panels, wherein the first window and the second window are distinct.

5. The retail package of claim 4 wherein the first window and the second window occupy a common panel.

6. The retail package of claim 5 wherein the second window occupies the bottom-front corner and at least of the left-front corner and the right-front corner.

7. The retail package of claim 1 wherein the polymer sheet comprises a polymer selected from the group consisting of polypropylene, polyethylene terephthalate (PET), modified PET and combinations thereof.

8. The retail package of claim 1 wherein the paperboard has a first Bending Resistance and the polymer sheet has a second Bending Resistance, wherein the second Bending Resistance is at least 30 percent, more preferably at least 40 percent, and even more preferably at least 50 percent, of the first Bending Resistance.

9. The retail package of claim 1 comprising a recloseable access opening configuration, wherein the container is configured such that no cut edges of the polymer sheet project or extend away from the paperboard by opening of the container via the recloseable access opening configuration.

10. The retail package of claim 11 having an opening cutout on a panel that is not occupied by a window.

11. The retail package of claim 10 wherein the opening cutout defines an opening flap, wherein lifting the flap away from the unoccupied panel creates an access opening in the unoccupied panel, providing access to interior space within the container.

12. The retail package of claim 11 wherein the unoccupied panel and/or the opening flap are provided with a recloseability feature whereby following opening, the flap may be closed over the opening and held in a closed position by the recloseability feature.

13. The retail package of claim 4 wherein the first window and/or the second window is/are present on a facing panel of the package.

14. The retail package of claim 13 wherein a plurality of individually consumable products (200) are disposed inside the container and may be seen from outside the package, through one or both of the first window and the second window.

15. The retail package of claim 14 wherein the plurality of individually consumable products is present in an orderly stack and at least a portion of the stack may be seen from outside the package, through one or both of the first window and the second window.

16. The retail package of claim 14 wherein one or more of the plurality of individually consumable products is/are individually packaged.

17. The retail package of claim 14 wherein the individually consumable products are absorbent articles.

18. The retail package of claim 17 wherein the absorbent articles are feminine hygiene pads.

19. The retail package of claim 17 wherein the absorbent articles are each folded into a substantially flat configuration having two opposing major surfaces.

20. The retail package of claim 17 wherein the absorbent articles are arranged in at least one orderly stack with major surfaces of individual articles facing adjacent major surfaces of adjacent articles in the stack, and the stack is at least partially visible through one or both of the first window and the second window.
